(19) United States
(12) Patent Application Publication
(10) Pub. No.: US 2026/0102131 A1
(43) Pub. Date: Apr. 16, 2026

(54) SPIRAL SPECT WITH FLEXIBLE AND ADAPTIVE DETECTOR ARRAY

(71) Applicant: Siemens Medical Solutions USA, Inc., Malvern, PA (US)

(72) Inventors: Alexander Hans Vija, Evanston, IL (US); Andrew Scheffel, Grayslake, IL (US)

(21) Appl. No.: 19/115,522

(22) PCT Filed: Sep. 15, 2023
(86) PCT No.: PCT/US2023/032827

§ 371 Date: Mar. 26, 2025

Related U.S. Application Data
(60) Provisional application No. 63/478,519, filed on Jan. 5, 2023.

(51) Int. Cl. A61B 6/42; A61B 6/00; A61B 6/03

(52) U.S. Cl. CPC A61B 6/4266; A61B 6/037; A61B 6/4458; A61B 6/54

(57) ABSTRACT

An imaging system for gamma ray imaging includes a gantry with an opening configured to receive a patient to be imaged and a plurality of imaging arms. The arms each include a detector head having an imaging face that receives and detects gamma radiation from the patient and an arm coupled to the gantry with a plurality of actuators configured to move the detector head radially within the gantry, tilt the imaging face relative to an axis of the arm, and to adjust spacing between detector heads on adjacent imaging arms. A processor is configured to coordinate movement of the imaging arms such that together the imaging faces of the plurality of imaging arms selectively form one of a plurality of predetermined imaging surfaces within the gantry. One of the predetermined imaging surfaces is a flat plane with uniform spacing of the plurality of detector heads suitable for planar imaging. Other surfaces are suitable for tomographic imaging.

### CROSS REFERENCE TO RELATED APPLICATIONS

This application claims the benefit of U.S. Provisional Application Ser. No. 63/478,519 filed Jan. 5, 2023, which is incorporated herein by reference in its entirety.

### TECHNOLOGY FIELD

This application relates generally to the field of emission imaging and more specifically to SPECT imaging where tomographic imaging and planar imaging are both used in imaging patients.

### BACKGROUND

Single-photon emission computed tomography (SPECT) is a nuclear medicine (NM) tomographic imaging technique using gamma rays emissions from patient tissue.

Standard computed tomography (CT) image images the transmission of radiation (e.g., x-rays). SPECT and CT imaging are commonly performed in a single imaging system, allowing flexible radiological scans of patients from a single machine. Much of the current clinically SPECT/CT systems deployed in industry use large field of view (FOV) gamma cameras, such that the machine occupies a large footprint (e.g., in excess of 20 m²). For example, Symbia Pro. Specta SPECT/CT system, which is a room-size optimized system, requires a 5.6 m×3.7 m minimum room size. The fundamental technical problem is with the use of the Anger Camera (a gamma camera using a NaI scintillator plate and a photomultiplier tube) using multi-channel collimator to form a projection image (2D) of the 3D activity distribution at some viewing angle with respect to the patient. If only planar scintigraphy is clinically indicated, then a few angular exposures may suffice for a scan. If tomography is needed, then multiple exposures must be acquired sampling at least a 180 deg arc around the long axis of the patient. The axial dimension (parallel to the patient axis) of the large-FOV anger camera is typically about 40 cm and the transaxial (meaning a non-radial axis normal to the axial direction) dimension is about 50 cm, enabling anatomically relevant features to be assessed in one scintigraphy exposure. Such a detector is large (2000 cm²), must generally be shielded against stray gamma radiation, and weighs more than 200 kg. A supporting gantry is correspondingly designed to enable the needed rotational and translational motion of the detector(s). Other designs using Room Temperature Semiconductor Detector (RTSD), such as Cadmium Zinc Telluride (CZT) crystals (instead of a photomultiplier), need a multi-channel collimation resulting still in large room size requirements. Namely, to enable a multitude of encompassing detector elements to obtain sufficient tomographic viewing coverage, swiveling motion is generally needed in addition to rotational and translational motion. Other designs with smaller footprints, including dedicated organ cameras, use a stationary pinhole based tomographic imaging approach with a fixed arc and distribution of pinholes, and are only able to image the specific (yet incomplete) organ in question (e.g., a heart) and do not fully encompass the entire torso.

These traditional designs utilize gamma detectors that are generally large and expensive, so that it is impractical to add more than a handful of detectors. Furthermore, these detectors tend to be relatively large in the axial direction to increase particle counts to ensure quality images. The low number of detectors and the long axial length means that while spiral imaging is theoretically possible, it is impractical in a medical setting.

### SUMMARY

Described herein are systems and methods for capturing gamma ray images of a patient by utilizing imaging arms with several degrees of freedom to reposition and rearrange gamma ray cameras to form different shapes of imaging surfaces from the faces of the cameras. These arms allow different imaging types to be captured with a single set of imaging arms, including planar imaging and spiral imaging.

According to one embodiment, an imaging system comprises a gantry with an opening configured to receive a patient to be imaged and a ring configured to orbit the patient under processor control, a plurality of detector heads having an imaging face configured to receive and detect gamma radiation from the patient, and a plurality of extendable radial arms. The radial arms are each coupled to the ring at a plurality of locations along the ring and configured to translate one of the plurality of detector heads radially and to orient the imaging face of each detector head under processor control. A processor is configured to coordinate movement of the plurality of extendable radial arms such that together the imaging faces form a plurality of predetermined imaging surfaces within the gantry. At least one of the predetermined imaging surfaces comprises a flat plane with uniform spacing of the plurality of detector heads.

According to another embodiment, an imaging system comprises a gantry with an opening configured to receive a patient to be imaged and a plurality of imaging arms. Each arm comprises a detector head having an imaging face that receives and detects gamma radiation from the patient and an arm coupled to the gantry with a plurality of actuators. The plurality of actuators are configured to move the detector head radially within the gantry, tilt the imaging face relative to an axis of the arm, and to adjust spacing between detector heads on adjacent imaging arms. A processor is configured to coordinate movement of the plurality of imaging arms such that together the imaging faces of the plurality of imaging arms selectively form one of a plurality of predetermined imaging surfaces within the gantry. At least one of the predetermined imaging surfaces comprises a flat plane with uniform spacing of the plurality of detector heads.

According to one aspect of some embodiments, at least one of the predetermined imaging surfaces comprises two orthogonal flat planes with uniform spacing of the plurality of detector heads within each plane. According to another aspect of some embodiments, at least one of the predetermined imaging surfaces comprises an arced surface with uniform spacing of the plurality of detector heads within each plane. According to another aspect of some embodiments, the spacing of the plurality of detector heads within the flat plane lacks substantial gaps. According to another aspect of some embodiments, each imaging face is sized with a range of 7 to 12 cm by 10 to 16 cm. According to another aspect of some embodiments, at least one predetermined imaging surface comprises a subset of the plurality of detector heads and another subset of the plurality of detector heads is retracted.

According to another aspect of some embodiments, the processor is further configured to control motion of the ring and to capture spiral images of the patient. According to another aspect of some embodiments, the processor is further configured to control motion of the plurality of extendable radial arms such that the plurality of detector heads orbit in a non-circular orbit. According to another aspect of some embodiments, the processor is further configured to capture stationary planar images of the patient. According to another aspect of some embodiments, the processor is further configured to capture tomographic images of the patient.

### FIGURES

The accompanying drawings, which are incorporated in and form a part of the specification, illustrate the embodiments of the invention and together with the written description serve to explain the principles, characteristics, and features of the invention. In the drawings:

FIG. 1 is a schematic illustration of an extendable radial arm and detector head for use in some embodiments;

FIGS. 2a-2b are schematic illustrations of an extendable radial arm and detector head for use in some embodiments;

FIG. 3 is a schematic illustration of an extendable radial arm and detector head for use in some embodiments;

FIGS. 4a-4b are schematic illustrations of an extendable radial arm and detector head for use in some embodiments;

FIGS. 5a-5d are axial views of exemplary gantry and arm arrangements for use in some embodiments;

FIGS. 6a-6c are axial views of exemplary arm and detector arrangements for use in some embodiments;

FIG. 7 is a system diagram for an imaging system that can be used in some embodiments; and

FIG. 8 is a perspective view of an exemplary imaging system that can be used in some embodiments.

### DESCRIPTION

This disclosure is not limited to the particular systems, devices and methods described, as these may vary. The terminology used in the description is for the purpose of describing the particular versions or embodiments only, and is not intended to limit the scope.

As used herein, the terms “algorithm,” “system,” “module,” or “engine,” if used herein, are not intended to be limiting of any particular implementation for accomplishing and/or performing the actions, steps, processes, etc., attributable to and/or performed thereby. An algorithm, system, module, and/or engine may be, but is not limited to, software, hardware and/or firmware or any combination thereof that performs the specified functions including, but not limited to, any use of a general and/or specialized processor in combination with appropriate software loaded or stored in a machine-readable memory and executed by the processor. Further, any name associated with a particular algorithm, system, module, and/or engine is, unless otherwise specified, for purposes of convenience of reference and not intended to be limiting to a specific implementation. Additionally, any functionality attributed to an algorithm, system, module, and/or engine may be equally performed by multiple algorithms, systems, modules, and/or engines, incorporated into and/or combined with the functionality of another algorithm, system, module, and/or engine of the same or different type, or distributed across one or more algorithms, systems, modules, and/or engines of various configurations.

Embodiments of a SPECT/CT scanner address one or more challenges by including a plurality of movable detectors in a circumferential gantry that surrounds a patient on a slidable bed. Detectors can be moved circumferentially (independently or as a group, in some embodiments) along the gantry to set up a given detection/image. Meanwhile, the patient is positioned axially by sliding a bed (typically aligned parallel to the sagittal plane) through the plane of the gantry (aligned parallel to the transverse plane of the patient). In some embodiments, the bed can be translated in three directions. In addition to moving along a circumferential track in the gantry, detectors can be independently positioned radially by a linear actuator under processor control to place the imaging head selectively closer to the patient to improve collimation resolution and gamma ray collection. Furthermore, the detector head (detector placed close to the patient on the distal end of the arm) can swivel (under processor control) relative to the linear actuator. In some embodiments, the detector head and arm include an additional degree of freedom to aid is proper placement and orientation of the detector head. This allows off-radius focusing of one or more detectors. This can be used to focus tomographic imaging at a location in the patient at the center of the gantry or off-center. Further, this arrangement can be used to selectively place the planes of individual detector heads in the same plane to take planar images. Thus, with just a few radially extendable gamma detectors, a single machine can be used for 2D planar imaging or tomography.

U.S. Pat. No. 9,213,110 describes a SPECT system that uses a series of radially extendable detector columns that can be attached to a gantry at predefined circumferential positions, allowing them to orbit at a fixed angular distance from one another. These columns have a gamma detector that is elongated in the patient's axial direction, but very narrow in the transaxial direction (using <4×4 cm detector elements arranged in a row of up to seven elements). This narrow shape is necessary to allow the detector head of each column to tilt along an axis that parallels the patient and gantry plane axes inside a narrow, elongated housing. Tilting the detector head is used to adjust the field of view of each detector head.

While the form factor of the disclosed detector columns of U.S. Pat. No. 9,213,110 necessitates transaxially narrow detector heads, embodiments disclosed herein use a different approach, employing detector heads that are intentionally wider than those heads. The advantage of such an arrangement is that heads can be slid radially and tilted to form a flat row of detector plates for planar imaging. Exemplary detector heads used in some embodiments utilize a more square-like aspect ratio, such as a 8-10 cm×12-14 cm detector. These less elongated (in the axial direction) and wider (in the transaxial direction) detectors lend themselves to certain advantages. A shorter axial dimension results in less multiplexing for axial slices of anatomy, which makes spiral imaging possible. Meanwhile, a wider transaxial dimension allows for more gamma ray collection within a given anatomical slice, for faster imaging of a given axial location. It also facilitates placing adjacent detectors with little to no gap between the detectors. When detector arms are radially extended such that the detector pivot points are in a line, the detector heads can be tilted to form a single plane with minimal gaps. This is suitable for 2-D planar imaging. Thus, by using wider, less elongated detector heads, detectors can be tiled for planar imaging or formed into a circle or elliptical shape (or an arc thereof) for use in 3D tomography. By configuring the detector heads into an arc (or orthogonal planes), spiral (by translating the patient axially) or 4D imaging is possible.

These more rectangular gamma detectors for the detector heads can be achieved, for example, by using the 2-D tiling of gamma detectors/Compton cameras explained in U.S. Pat. No. 11,647,973, which is incorporated herein in its entirety. These detectors include a collimating pinhole plate, a scintillating crystal, and a plurality of solid state photo detectors or photo multiplier tubes. The detector heads can be divided into an array of tiles or formed as a single unit with a single collimation plate. It should be noted that any suitable configuration of collimation plate can be used, such as a grating with parallel holes or with slightly non-parallel holes that create a lensing focusing effect.

The collimator plate of a gamma detector includes a plurality of pinholes that may be parallel or multiplexed, focusing/magnifying. Wider detectors allow parallel or multiplexed/focusing collimator. Exemplary detector heads include an imaging face with a total of 8-10 cm by 12-14 cm of collimator, with a scintillator and an array of solid-state photodetectors. Exemplary detector heads are 7-12 cm by 10-16 cm in some embodiments.

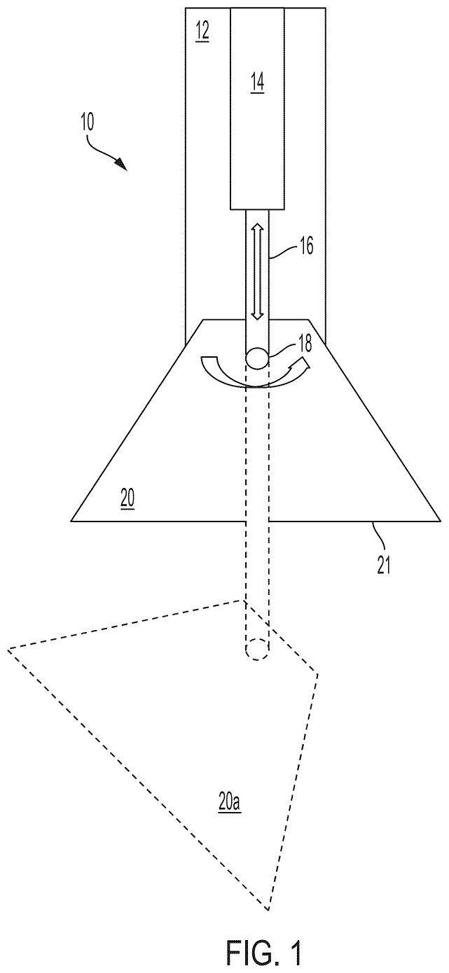

FIG. 1 is a block diagram illustrating exemplary features for an extendable radial arm that can support a detector head and provide at least two degrees of freedom (in this case, radial distance and one degree of tilt). This radial arm is coupled to a ring within the gantry of the imaging device. The arms extend inward from the gantry in the center opening of the gantry toward the patient who lays on a bed that is cantilevered into the opening of the gantry. The ring can be rotated within the gantry to allow a detector head on the distal end of each radial arm to orbit a patient to collect gamma-ray radiation to image the patient's anatomy. Radial arm 10 includes a main body 12 that couples to the ring of the gantry (not shown but understood to be at the top of the page) and a linear actuator 14 that includes an extension rod 16 (piston or ball screw, for example) that can extend under processor control away from the proximal end of the arm (top of the page). An example of this extension is shown in dotted lines. Any suitable linear actuating mechanism can be used, including a linear motor, a motor that drives a ball screw, or a pneumatic or hydraulic piston. The linear actuator should be suitable for processor control, including encoding so that the processor can confirm the extension of the linear actuator. This allows precise radial distance control of the detector head by a processor. The linear actuator encompasses the appropriate drivers for the motion mechanism, as well.

At the distal end of rod 16, a pivot point 18 provides a pivoting axis for detector head 20. Detector head 20 can use any suitable gamma camera. Within a housing of detector head 20, a motor or other suitable drive mechanism can pivot detector head 20 relative to rod 16 at pivot point 18 under processor control. In the example of FIG. 1, a processor actuates linear actuator 14 to extend rod 16 radially, extending the distal end of the rod and thereby pivot point 18 (thereby reducing the radial distance from the center of the gantry as it extends). A motor, such as a stepper motor, within the housing of detector head 20 allows the processor to rotate the detector face 21 (base of the illustrative trapezoid) to angle the collimation plate of the detector head towards a desired target. This is shown in pose 20a. Thus, a processor controlling radial arm 10 can control the distal location and orientation of imaging face 21 of the detector relative to the proximal base of the radial arm that is coupled to the gantry.

While main body 12 and rod 16 are shown with one being substantially wider than the other, any suitable arrangement can be used. For example, the drive components of the linear actuator 14 could be housed inside of a housing that moves radially, rather than residing in the fixed portion of the radial arm as shown in FIG. 1. Furthermore, a shroud can be placed around any part of the radial arm to keep the mechanical and electrical components safe from the hospital environment. Note that these illustrative embodiments are not to scale, and the arms may be wider/narrower or longer/shorter depending on design needs. Similarly, the exact shape of the detector head may be quite different without changing the illustrative functionality.

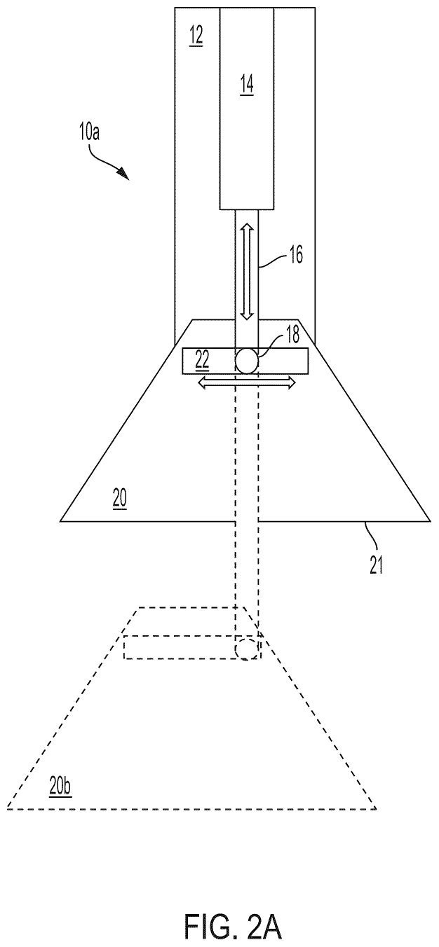

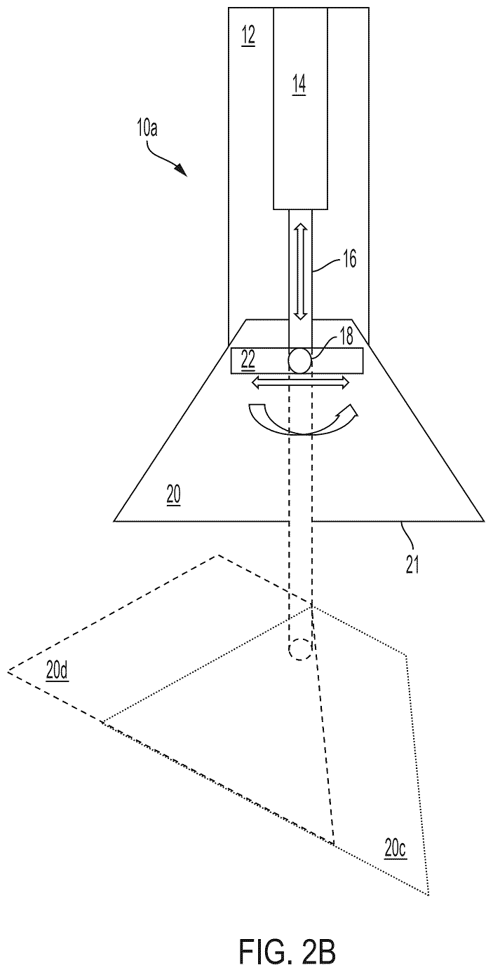

FIG. 2a shows an alternative embodiment of a radial arm 10a with an additional degree of freedom to translate the detector head relative to a centerline of the linear actuator. Like radial arm 10, radial arm 10a includes a body 12 and an actuator 14, along with an extension rod 16 and a pivot point 18. However, this embodiment adds a sliding slot 22 within detector 20. This allows the body of detector 20 to be translated right or left relative to pivot point 18. This translation can be done under processor control using a linear actuator or motor with rack and pinion gearing. Pose 20b provides an example of how a processor might orient the imaging face of detector 20. In this example, rod 16 is extended radially and the pivot point is translated laterally relative to the body of detector head 20. This allows extension, tilt, and offset for more precise placement of the imaging face 21. Specifically, slot 22 provides an additional degree of freedom to allow the center of the imaging face to be shifted laterally from a centerline of radial arm 10a. This allows a processor to adjust spacing of imaging faces on adjacent radial arms.

FIG. 2b shows two additional poses 20c and 20d. In these poses, the linear actuator is extended and the detector head pivots about pivot point 18 and slides relative to the pivot point using slot 22. This can be accomplished using a drive in linear actuator 14 (such as a motor and ball screw) to extend the rod 16, using a motor within detector head 20 to pivot the body of the detector head relative to pivot point 18, and using an actuator or motor in detector head 20 to provide additional translation of the extents of imaging face 21 relative to pivot point 18.

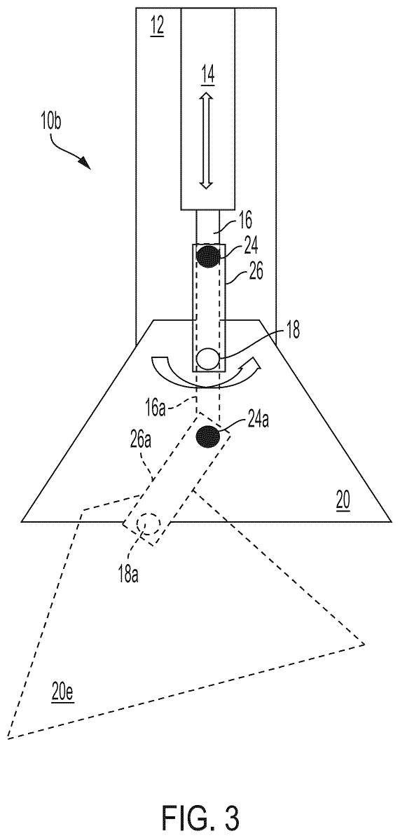

FIG. 3 shows another embodiment of a radial arm 10b that includes an additional degree of freedom compared to radial arm 10. In this example, body 12 and actuator 14 are similar, but rod 16 couples to an intervening member 26 that is coupled to the distal end of rod 16 via pivot 24. Intervening member includes pivot point 18 at its distal end, allowing a shift of the body of detector head 20 relative to the pivot point 24. Pivot point 24 can be managed via a stepper motor within (or mounted to) member 26, rod 16, or in detector head 20 (and may be driven via a belt or gearing). Alternatively, intervening member 26 can be coupled to two linear actuators (instead of a single actuator as shown). If member 26 is coupled to two actuators at offset pivot points (instead of a single pivot point 24, any difference in actuator length will cause an angular difference between rod 16 and member 26. In some embodiments, intervening member 26 is inside the body of detector head 20, allowing a more compact arrangement. In some embodiments, pivot point 24 may be off-center in detector head 20 to increase the offset effect.

Pose 20e illustrates how intervening member 26 can be used to change both the orientation of the face of the detector head and the offset of the face relative to the centerline of rod 16. In pose 20e, “a” will be added to each component to explain how they result in pose 20e. Rod 16a extends causing pivot points 24a to extend radially toward the patient. Intervening body 26a pivots relative to rod 16a, causing pivot points 18a to be offset relative to the center line actuator. The detector head pivots relative to position, causing the detector head to orient in pose 20e. This results in an extension (via rod 16a), a tilt (via pivot point 18a), and an offset from the centerline of the linear actuator 14 (via intervening body 26a). Intervening body 26 and additional pivot point 18 provides an additional degree of freedom to allow the center of the imaging face to be shifted laterally from a centerline of radial arm 10a. This allows a processor to adjust spacing of imaging faces on adjacent radial arms.

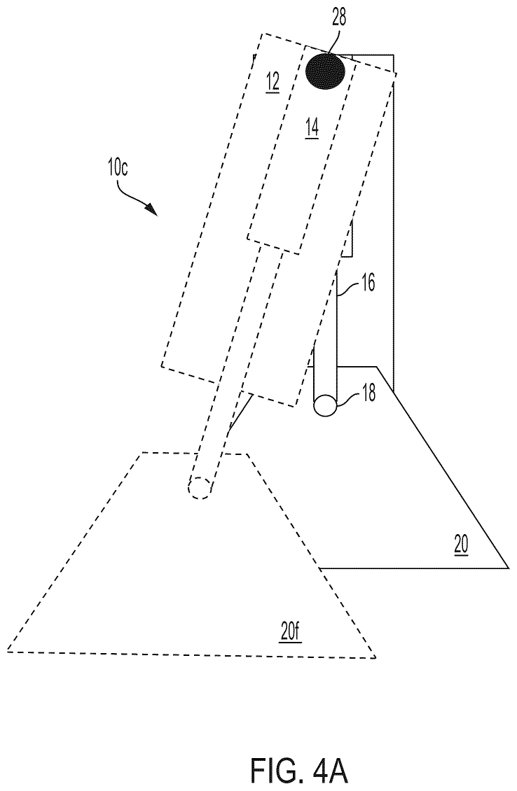

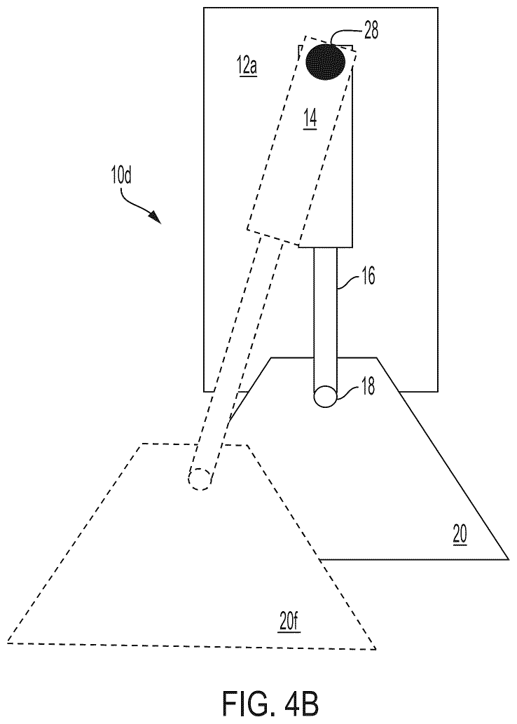

FIG. 4a illustrates an additional way of adding another degree of freedom in radially extending arm 10c. Pivot point 28 at the base of actuator 14 provides a degree of freedom to allow face 21 to shift relative to the normal center line. This pivot point can be driven by a motor or can be the natural result of two linear actuators in place of actuator 14 where the bases of the two mini racers have offset pivot points at the proximal end and share a common pivot point 18. Any difference in the length of the two actuators will naturally cause the pivot point 18 to shift left or right. And some embodiments, such as that shown in FIG. 4b, housing 12 of the arm can be fixed with a hollow opening large enough to allow left or right shift of pivot point 18 to occur without interference between body 12 and actuator 14. In some embodiments, housing 12 pivots with actuator 14. FIG. 4a shows a pose 20f, where actuator 14 extends allowing the face of detector 20 to move closer to the patient and be offset from the normal center line. FIG. 4B shows a similar embodiment of an arm 10d where housing 12a is fixed but with an opening large enough to allow pose 20f to be achieved without interference between housing 12a and rod 16. Pivot provides an additional degree of freedom to allow the center of the imaging face to be shifted laterally from a radial centerline of radial arm 10c. Note that these preceding examples place the radial arm along a radial line of an imaging gantry (not shown, but assumed to be at the top of the page.) Thus, the radial centerline of the radial arm is directly down the page, making an imaginary line from the gantry circumference to the center of the gantry. This allows a processor to adjust spacing of imaging faces on adjacent radial arms.

The reason for these additional degrees of freedom in the preceding embodiments is generally to allow an offset of the detector imaging face relative to the natural center line of a radially extending arm under processor control. This offset allows a uniform spacing between the faces of adjacent detector heads for non-circular imaging arrangements when different radially extending arms have different radial extensions. In the case of planar imaging, this can be an important feature because 2D imaging benefits from a uniform arrangement of detector heads without substantial gaps between adjacent detectors. The embodiments in FIGS. 2a-4b illustrate radial detector arms with three degrees of freedom - radial extension (such as via a linear actuator), tilt of the detector head body relative to the axis of the radial arm (such as via a rotational actuator), and one additional degree that varies by embodiment (which allows an adjustment of the spacing of adjacent detector head imaging faces). Note that adjusting spacing between adjacent detector heads is not meant to refer to adjusting the radial extension, which naturally changes spacing as the ends of the arms will be closer to one another the closer the end moves toward the center point. Instead, adjusting the spacing refers to shifting the center of the imaging face from the radial line of the gantry while allowing the radial extension and the tilt (orientation) of the imaging face.

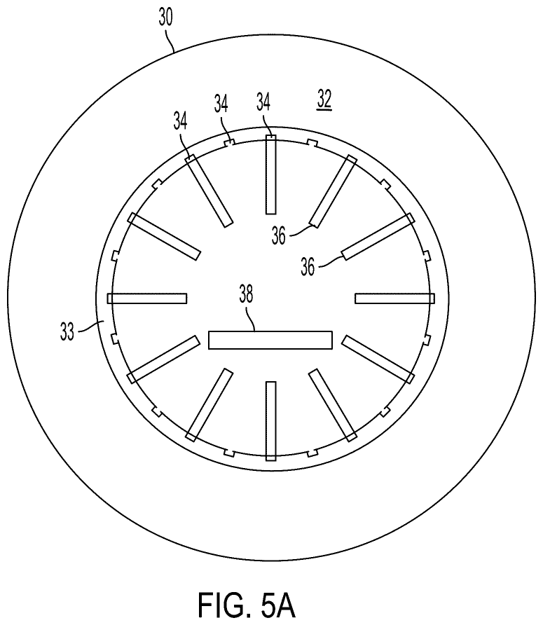

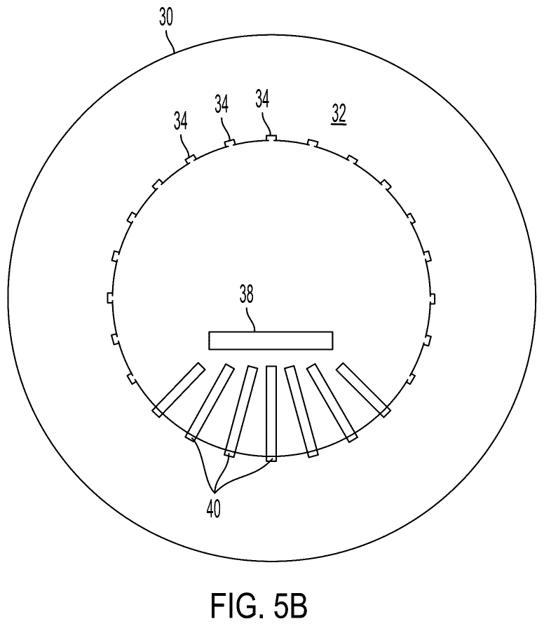

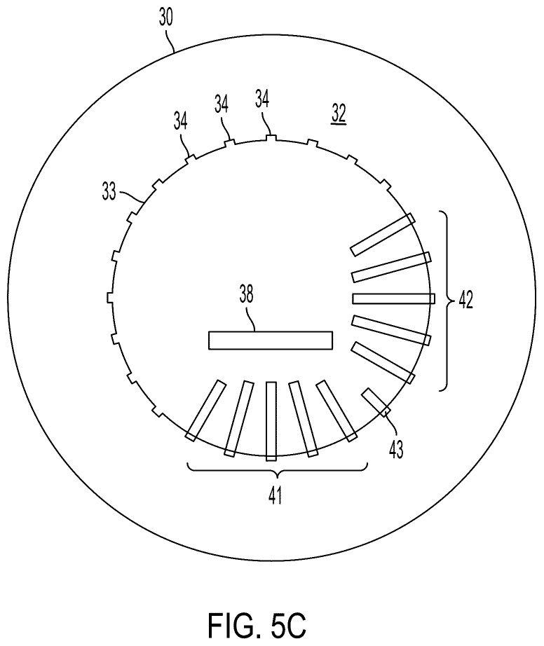

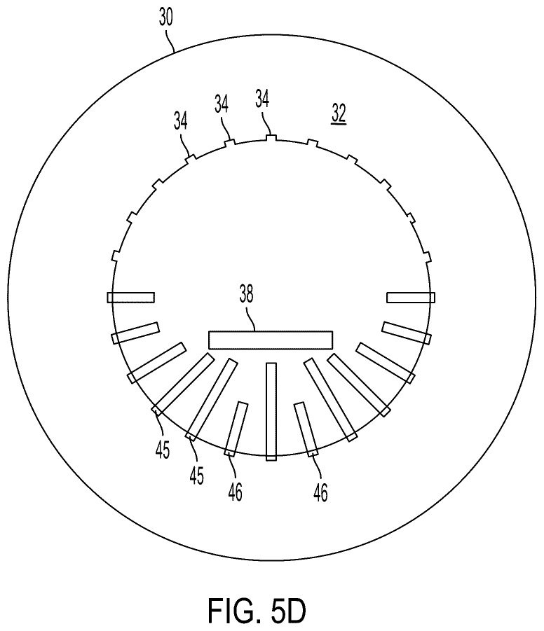

FIG. 5A shows an axial view of an imaging system 30. Ring-shaped gantry 32 includes an inner ring 33 that can revolve relative to the gantry housing. This allows detector heads coupled to this inner ring 33 to orbit the patient during tomographic imaging, or for placement of stationary 2-D views. In this example, inner ring 33 includes a plurality of available detector position slots 34. Radial arms 36 can be removable and selectively placed into these positions before imaging. These arms can be any of those shown in FIGS. 1-4b. In this example, twelve arms are placed in every other slot in of the twenty-four possible slots 34. Radial arms can be coupled to the slots at any of these positions. Slots 34 can include mechanical and electrical coupling components suitable for removably holding the radial arms and for providing power, control signals, and image data pathways to the arms and their detector heads. In some embodiments, slots 34 or fixed positions in ring 33 that allow selective mounting of radial arms. In some embodiments, slots 34 are movable relative to one another within ring 33. Patient bed 38 resides near the middle of the opening, allowing a patient to be selectively placed within the ring. Bed 38 can slide and translate in at least the vertical direction (and left/right in some embodiments.)

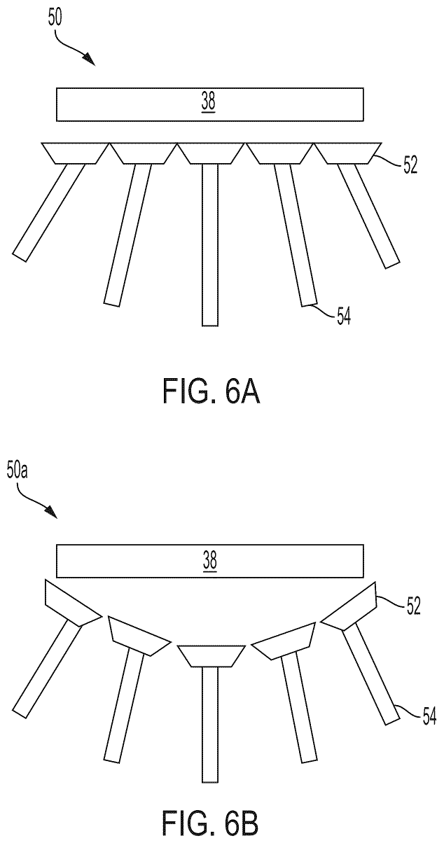

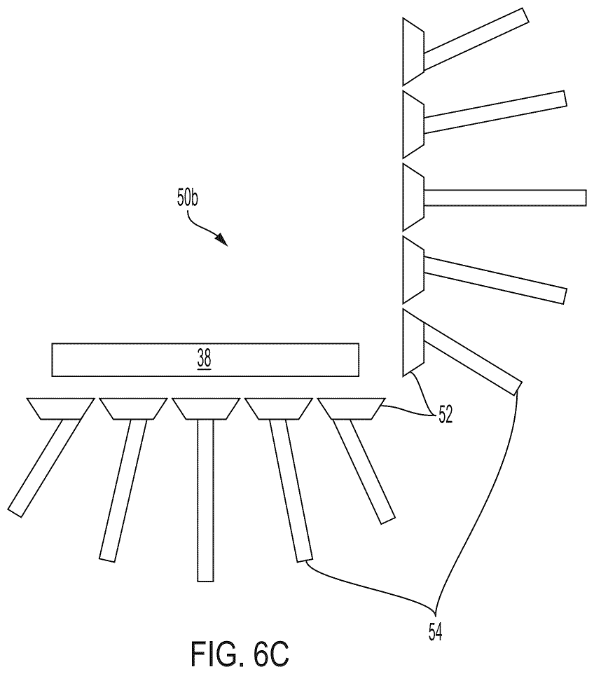

FIG. 5b shows an exemplary arrangement of seven radial arms 40 within slots 34. In this example, the radial length of each arm 40 has been adjusted for planar imaging. To illustrate this length, detector heads are not illustrated, but FIGS. 6a-c provide additional illustrations of how detector heads can be arranged. Not that the spacing between the distal ends of arms 40 varies when the distal ends are arranged in a line. This can be resolved with the additional degrees of freedom illustrated in FIGS. 2-4b, which allows the faces of the detector heads to be translated to selectively change or remove the gaps under processor control.

FIG. 5c shows a related example where arms are arranged in slots 34 in a few different groups. Group 41 includes five radial arms arranged for planar imaging underneath the patient. Group 42 is arranged and extended to provide planar imaging at an orthogonal direction on the patient side. Meanwhile arm 43 has been fully retracted by the processor to prevent mechanical interference between detector heads. The detector heads attached to the distal ends of arms 42 and 43 can be tilted to provide two orthogonal planar imaging surfaces.

FIG. 5d shows an example where the radial arms form half of an elliptical imaging surface. Notice that the ends of the arms form a non-circular arc facing the patient. Detector heads on the distal ends of these arms can form a generally continuous half-ellipse imaging surface. In some embodiments, the arms extend and retract automatically under processor control as the ring of gantry 32 orbits the patient such that the detector heads follow an elliptical imaging surface around the patient (where only half exists at a given time). In this example, some arms, such as arms 45 are extended to take part in the imaging surface, while some arms, such as arms 46, are fully retracted to avoid mechanical interference. This is especially important in areas where arms are almost fully extended.

FIGS. 6a-6c are axial diagrams of different arrangements of imaging surfaces that can be created under processor control, such as in the examples shown in FIGS. 5a-5d. In arrangement 50, arms 54 are extended and detector heads 52 are further translated to form a planar imaging surface. In this example, the heads are translated to essentially touch so that this forms a continuous planar surface without substantial gaps. While gaps should be minimized, in the context of a planar imaging surface, gaps are insubstantial if they are less than 5 mm.

In arrangement 50a, detector heads 52 are formed into an arced imaging surface with small, uniform gaps. These gaps can provide spacing to avoid mechanical interference, especially where the arms may move during the gantry orbit to maintain an imaging surface shape relative to the patient frame of reference during orbit.

In arrangement 50b, arms 54 are extended and detector heads 52 are further translated to form two planar imaging surfaces.

### Medical Imaging System Architecture

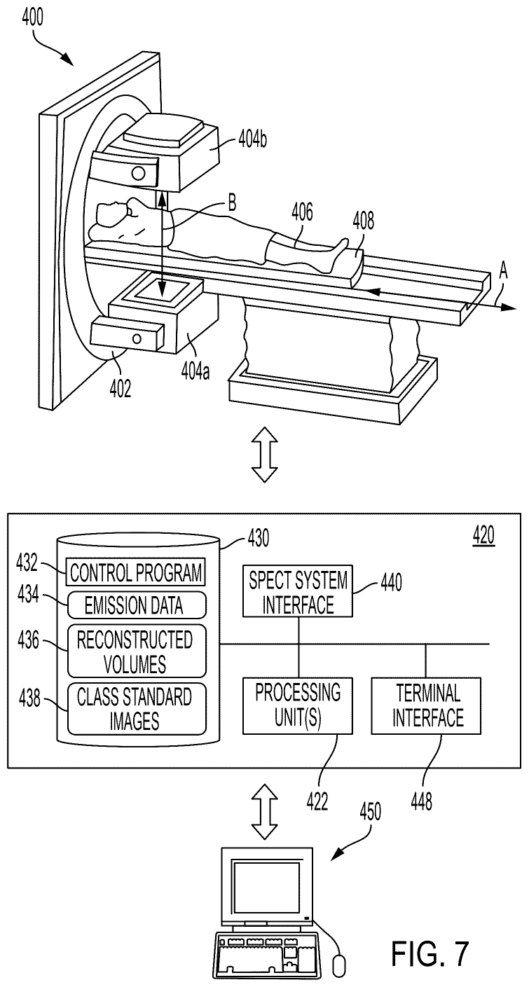

FIG. 7 illustrates SPECT system 400 which may implement process 300 as described above. System 400 includes gantry 402 to which two or more gamma cameras 404a, 404b are attached, although any number of gamma cameras can be used. A detector within each gamma camera detects gamma photons (i.e., emission data) emitted by a radioisotope within patient or phantom 406 on bed 408.

Bed 408 is capable of moving patient or phantom 406 along axis A and/or axis B. At respective bed positions (i.e., imaging positions), a portion of patient 406 is positioned between gamma cameras 404a, 404b in order to capture emission data from that body portion. Gamma cameras 404a, 404b may include multi-focal cone-beam collimators or parallel-hole collimators as is known in the art. Cameras 404a and 404b are merely illustrative and their mechanical arrangements can be understood in some embodiments as using the mechanisms discussed in FIGS. 1-6c.

Cameras generally include a collimator, a scintillation crystal, and a light sensor array. Scintillation crystals may comprise a thallium-doped sodium iodide crystal that generates light photons in response to gamma radiation received from the patient.

Conventionally, a radioactive isotope is administered to the patient. The radioactive isotope emits gamma photons while present in the patient and these gamma photons subsequently exit the patient. Gamma photons are collimated by the collimator in each camera/detector head to define their line-of-response and to filter out scattered or stray gamma radiation. The collimated photons are received at various locations in the scintillation crystal, which converts the gamma radiation into light photons which may be detected by the sensor array

The sensor array may comprise an array of photomultiplier tubes (PMTs). A typical PMT of sensor array may include a semi-transparent photocathode, a focusing grid, dynodes, and an anode. The sensor array converts light photons emitted by scintillation crystal into electronic signals representing the number of light photons collected. A signal processing unit receives the electronic signals from sensor array and processes the electronic signals to generate an image of the patient anatomy. Solid-state photo detectors can also be used in the sensor array.

Control system 420 may comprise any general-purpose or dedicated computing system. Accordingly, control system 420 includes one or more processing units 422 configured to execute processor-executable program code to cause system 420 to operate as described herein, and storage device 430 for storing the program code. Storage device 430 may comprise one or more fixed disks, solid-state random access memory, and/or removable media (e.g., a thumb drive) mounted in a corresponding interface (e.g., a USB port).

Storage device 430 stores program code of system control program 432. One or more processing units 422 may execute system control program 432, in conjunction with SPECT system interface 440, to control motors, servos, and encoders to cause gamma cameras 404a, 404b to rotate along gantry 402 and to acquire two-dimensional emission data (i.e., projection images) at defined imaging positions during the rotation. The acquired data 434 may be stored in memory 430. Control program 432 may also be executed to reconstruct volumes 436 from emission data 434 as is known.

Control program 432 may also be executed to cause control system 420 to perform process 300, including acquisition of class standard images 438, administration of comparisons between a test image and each of class standard images 438, and evaluation of a test component based on the comparisons.

Terminal 450 may comprise a display device and an input device coupled to system 420 via a terminal interface 448. Terminal 450 may display any of two-dimensional emission data 434 and reconstructed volumes 436. Terminal 450 may also display a test image alongside a class standard image 438, and receive user input indicating a choice of one displayed image over the other. In some embodiments, terminal 450 is a separate computing device such as, but not limited to, a desktop computer, a laptop computer, a tablet computer, and a smartphone.

Each of component of system 400 may include other elements which are necessary for the operation thereof, as well as additional elements for providing functions other than those described herein. It is understood by those familiar with the art that the system described herein may be implemented in hardware, firmware, or software encoded (e.g., as instructions executable by a processor) on a non-transitory computer-readable storage medium.

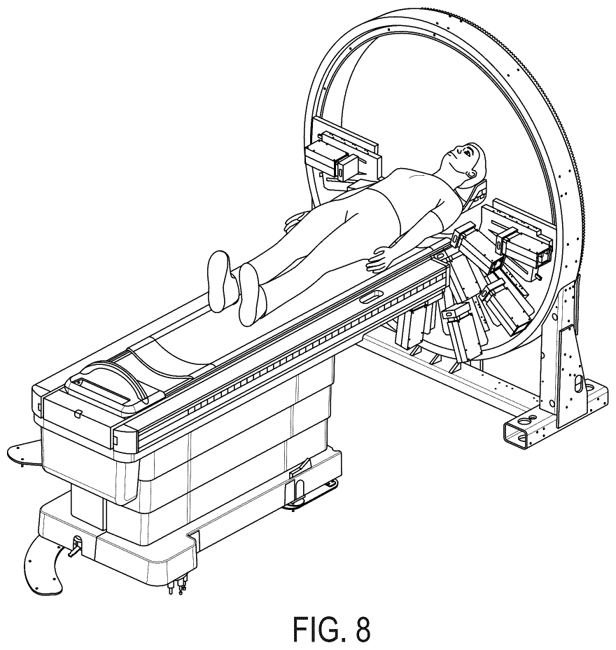

FIG. 8 is a 3D illustration of the imaging system of FIG. 7 using a plurality of radial arms and distal detector heads consistent with embodiments disclosed herein.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

Aspects of the present technical solutions are described herein with reference to flowchart illustrations and/or block diagrams of methods, apparatuses (systems), and computer program products according to embodiments of the technical solutions. It will be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present technical solutions. In this regard, each block in the flowchart or block diagrams can represent a module, segment, or portion of instructions, which includes one or more executable instructions for implementing the specified logical function(s). In some alternative implementations, the functions noted in the blocks can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

A second action can be said to be “in response to” a first action independent of whether the second action results directly or indirectly from the first action. The second action can occur at a substantially later time than the first action and still be in response to the first action. Similarly, the second action can be said to be in response to the first action even if intervening actions take place between the first action and the second action, and even if one or more of the intervening actions directly cause the second action to be performed. For example, a second action can be in response to a first action if the first action sets a flag and a third action later initiates the second action whenever the flag is set.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

Independent of the grammatical term usage, individuals with male, female or other gender identities are included within the term.

The following is a list of non-limiting illustrative embodiments disclosed herein:

Illustrative embodiment 1. An imaging system comprises a gantry with an opening configured to receive a patient to be imaged and a ring configured to orbit the patient under processor control, a plurality of detector heads having an imaging face configured to receive and detect gamma radiation from the patient, and a plurality of extendable radial arms. The radial arms are each coupled to the ring at a plurality of locations along the ring and configured to translate one of the plurality of detector heads radially and to orient the imaging face of each detector head under processor control. A processor is configured to coordinate movement of the plurality of extendable radial arms such that together the imaging faces form a plurality of predetermined imaging surfaces within the gantry. At least one of the predetermined imaging surfaces comprises a flat plane with uniform spacing of the plurality of detector heads.

Illustrative embodiment 2. An imaging system comprises a gantry with an opening configured to receive a patient to be imaged and a plurality of imaging arms. Each arm comprises a detector head having an imaging face that receives and detects gamma radiation from the patient and an arm coupled to the gantry with a plurality of actuators. The plurality of actuators are configured to move the detector head radially within the gantry, tilt the imaging face relative to an axis of the arm, and to adjust spacing between detector heads on adjacent imaging arms. A processor is configured to coordinate movement of the plurality of imaging arms such that together the imaging faces of the plurality of imaging arms selectively form one of a plurality of predetermined imaging surfaces within the gantry. At least one of the predetermined imaging surfaces comprises a flat plane with uniform spacing of the plurality of detector heads.

According to one of the preceding embodiments, at least one of the predetermined imaging surfaces comprises two orthogonal flat planes with uniform spacing of the plurality of detector heads within each plane. According to one of the preceding embodiments, at least one of the predetermined imaging surfaces comprises an arced surface with uniform spacing of the plurality of detector heads within each plane. According to one of the preceding embodiments, the spacing of the plurality of detector heads within the flat plane lacks substantial gaps. According to another aspect of some embodiments, each imaging face is sized with a range of 7 to 12 cm by 10 to 16 cm. According to one of the preceding embodiments, at least one predetermined imaging surface comprises a subset of the plurality of detector heads and another subset of the plurality of detector heads is retracted.

According to one of the preceding embodiments, the processor is further configured to control motion of the ring and to capture spiral images of the patient. According to one of the preceding embodiments, the processor is further configured to control motion of the plurality of extendable radial arms such that the plurality of detector heads orbit in a non-circular orbit According to one of the preceding embodiments, the processor is further configured to capture stationary planar images of the patient. According to one of the preceding embodiments, the processor is further configured to capture tomographic images of the patient.

1. An imaging system, comprising:
a gantry (32) with an opening configured to receive a patient to be imaged and a ring (33) configured to orbit the patient under processor control;
a plurality of detector heads (20) having an imaging face (21) configured to receive and detect gamma radiation from the patient;
a plurality of extendable radial arms (54) each coupled to the ring at a plurality of locations (34) along the ring and configured to translate one of the plurality of detector heads radially and to orient the imaging face of each detector head under processor control; and
a processor (422) configured to coordinate movement of the plurality of extendable radial arms such that together the imaging faces form a plurality of predetermined imaging surfaces within the gantry,
wherein at least one of the predetermined imaging surfaces comprises a flat plane (50) with uniform spacing of the plurality of detector heads.

2. The imaging system of claim 1, wherein at least one of the predetermined imaging surfaces comprises two orthogonal flat planes (50b) with uniform spacing of the plurality of detector heads within each plane.

3. The imaging system of claim 1, wherein at least one of the predetermined imaging surfaces comprises an arced surface (50a) with uniform spacing of the plurality of detector heads within each plane.

4. The imaging system of claim 1, wherein the spacing of the plurality of detector heads within the flat plane (50) lacks substantial gaps.

5. The imaging system of claim 1, wherein each imaging face (21) is sized with a range of 7 to 12 cm by 10 to 16 cm.

6. The imaging system of claim 1, wherein at least one of the predetermined imaging surfaces comprises a subset of the plurality of detector heads and another subset of the plurality of detector heads is retracted.

7. The imaging system of claim 1, wherein the processor is further configured to control motion of the ring and to capture spiral images of the patient.

8. The imaging system of claim 1, wherein the processor is further configured to control motion of the plurality of extendable radial arms such that the plurality of detector heads orbit in a non-circular orbit.

9. The imaging system of claim 1, wherein the processor is further configured to capture stationary planar images of the patient.

10. The imaging system of claim 1, wherein the processor is further configured to capture tomographic images of the patient.

11. An imaging system, comprising:
a gantry (32) with an opening configured to receive a patient to be imaged;
a plurality of imaging arms (54) each comprising a detector head (20) having an imaging face (21) that receives and detects gamma radiation from the patient and an arm (10a-10d) coupled to the gantry with a plurality of actuators (14, 18, 22, 24, 28) configured to move the detector head radially within the
gantry, tilt the imaging face relative to an axis of the arm, and to adjust spacing between detector heads on adjacent imaging arms; and
a processor (422) configured to coordinate movement of the plurality of imaging arms such that together the imaging faces of the plurality of imaging arms selectively form one of a plurality of predetermined imaging surfaces within the gantry,
wherein at least one of the predetermined imaging surfaces comprises a flat plane (50) with uniform spacing of the plurality of detector heads.

12. The imaging system of claim 11, wherein at least one of the predetermined imaging surfaces comprises two orthogonal flat planes (50b) with uniform spacing of the plurality of detector heads within each plane.

13. The imaging system of claim 11, wherein at least one of the predetermined imaging surfaces comprises an arced surface (50a) with uniform spacing of the plurality of detector heads within each plane.

14. The imaging system of claim 11, wherein the spacing of the plurality of detector heads within the flat plane (50) lacks substantial gaps.

15. The imaging system of claim 11, wherein each imaging face (21) is sized with a range of 7 to 12 cm by 10 to 16 cm.

16. The imaging system of claim 11, wherein at least one of the predetermined imaging surfaces comprises a subset of the detector heads of the plurality of imaging arms and another subset of the detector heads is retracted.

17. The imaging system of claim 11, wherein the processor is further configured to control motion of the gantry and to capture spiral images of the patient.

18. The imaging system of claim 11, wherein the processor is further configured to control motion of the plurality of imaging such that the plurality of detector heads orbit in a non-circular orbit.

19. The imaging system of claim 11, wherein the processor is further configured to capture stationary planar images of the patient.

20. The imaging system of claim 11, wherein the processor is further configured to capture tomographic images of the patient.
